(19) United States
(12) Patent Application Publication
(10) Pub. No.: US 2026/0049270 A1
(43) Pub. Date: Feb. 19, 2026

(54) MODULAR CROSS CONTAMINATION GUARD FOR AN IN VITRO EXPOSURE SYSTEM

(71) Applicant: Battelle Memorial Institute, Columbus, OH (US)

(72) Inventors: Amit Gupta, Dublin, OH (US); Patrick H. Keyes, Columbus, OH (US); Kent Hofacre, Worthington, OH (US); Janae Newswanger, Galloway, OH (US); Cassady Griffin, Columbus, OH (US)

(21) Appl. No.: 19/287,949

(22) Filed: Aug. 1, 2025

Related U.S. Application Data
(60) Provisional application No. 63/683,999, filed on Aug. 16, 2024.

(51) Int. Cl. C12M 1/12; C12M 1/00; C12M 3/00

(52) U.S. Cl. CPC C12M 37/04; C12M 23/38; C12M 23/44; C12M 23/46; C12M 23/48; C12M 23/50

(57) ABSTRACT

A contamination guard for an in vitro exposure system includes a plurality of well covers. Each of the plurality of well covers further includes a locking pin and a cover, the cover configured to cover a well in the in vitro exposure system to isolate the well from contamination; and a modular well plate cover, the modular well plate cover configured to align the plurality of well covers over a well plate of the in vitro exposure system.

### CROSS-REFERENCE TO RELATED APPLICATIONS

The present application claims the benefit of the filing date of U.S. Provisional Application Ser. No. 63/683,999, filed Aug. 16, 2024, the entire teachings of which application is hereby incorporated herein by reference.

### STATEMENT REGARDING FEDERALLY SPONSORED RESEARCH OR DEVELOPMENT

This invention was made with government support under contract number HHSN273201400015C awarded by Division of Translational Toxicology of the National Institutes of Health. The government has certain rights in the invention.

### TECHNICAL FIELD

The present disclosure relates generally to a modular cross contamination guard for an in vitro exposure system.

### BACKGROUND

An in vitro exposure system is a commercially available instrument which is designed to direct the exposure of cell cultures to airborne substances such as gases, aerosols, nano-particles, and more. During normal use, cell matrices contained in individual wells on the instrument's well plate are exposed to varying concentrations of the target analyte for specific durations before being removed from the instrument and collected for analysis. During the collection process, the entirety of the well plate and every cell matrix is left uncovered and exposed for extended periods of time. This long period of exposure allows for potential contamination as a result of gas exchange between wells or through deposited contaminants (dust, debris, etc.) as a result of manipulation of the wells during sampling. There exists a need to reduce cross contamination between replicate wells for in vitro exposure systems.

### BRIEF DESCRIPTION OF THE DRAWINGS

Reference should be made to the following detailed description which should be read in conjunction with the following figures, wherein like numerals represent like parts.

FIGS. 1A and 1B are cross sectional views illustrating a single well cover consistent with the present disclosure.

FIG. 2 is a cross sectional view illustrating a plurality of well covers loaded in a modular well plate cover, consistent with the present disclosure.

FIGS. 3-6 are cross sectional views illustrating the assembly and placement of a plurality of well covers loaded in a modular well plate cover, consistent with the present disclosure.

FIGS. 7A and 7B are a top perspective view (FIG. 7A) and a bottom perspective view (FIG. 7B) of a plurality of well covers loaded in a modular well plate cover, consistent with the present disclosure.

FIG. 8 is a top perspective view of a rack of modular well plate covers loaded with a plurality of well covers, consistent with the present disclosure.

FIG. 9 is an illustration of a modular well plate cover in position for assembly to a well plate.

FIG. 10 is a close-up illustration of two well covers in a modular well plate cover in position for assembly to a well plate.

FIG. 11 is a close-up illustration of two well covers inserted into a well plate.

FIG. 12 is an illustration of a well cover being removed from a well plate.

### DETAILED DESCRIPTION

Disclosed herein is a removable well plate cover and system to reduce cross contamination between instrument wells. To effectively reduce the cross contamination, the device should, at a minimum, isolate individual rows of replicate wells from adjacent rows of replicate wells of higher or lower analyte concentration. In addition, there is a need to cover cells rapidly upon conclusion of a test (i.e., opening of the instrument) to limit the exposure time. It is also desirable to allow easy access to individual rows of wells, during post-test analysis/collection, while continuing to isolate adjacent rows.

Disclosed herein is a device to reduce or prevent cross contamination between individual cell matrices in the in vitro exposure system by isolating test wells from one another as well as from the ambient environment. The disclosed device has the ability to cover wells individually (rather than by row), which may allow for more experimental flexibility. In addition, the device may be disposed within a modular well plate cover to allow for covering multiple wells simultaneously, which may increase throughput and further decrease exposure time, thereby reducing the possibility of cross contamination, while still providing the ability to cover wells individually.

As disclosed herein, the instrument wells are covered by individual stainless-steel disks, or covers, which may occlude the cell matrix below. Stainless steel well covers may be removably coupled into a modular well plate cover and may be affixed in placed via magnetic locking pins. In an embodiment, a single modular well plate cover may house an entire row of covers, which can be placed simultaneously. One example in vitro exposure system, the VITROCELL 24/48 Exposure System manufactured by VITROCELL SYSTEMS of Waldkirch, Germany, has wells arranged in rows of seven, and a single modular well plate cover may hold an entire row of seven covers, which can be placed simultaneously. Once in place, the magnetic locking pins can be removed, releasing the stainless covers, and allowing for individual manipulation of the wells.

In an embodiment, a modular well plate cover may include machined slots designed to house a series of covers. These covers are affixed to the modular well plate cover via magnetic locking pins, which keep the caps from disconnecting from the modular well plate cover but allow for enough freedom of motion for the caps to lay flat when set down. Once the user is ready for post-exposure collection of the wells, the modular well plate cover may be used, for example, to transfer a row's worth of caps onto the instrument well plate, where the caps are then lowered onto the tops of the wells below and occlude the opening to the cell matrices until ready to be accessed by the user. Additional modular well plate covers are used to cover each row of the well plate until the entirety of the plate is covered. Depending on the user's needs, an entire row of wells may be accessed by removing a modular well plate cover and its attached caps, or the caps may be disconnected from the modular well plate cover by removing the magnetic locking pins. In this later configuration, the caps are left on top of the wells once the modular well plate cover is removed and may then be removed individually as required by the user.

In the example of the VITROCELL in vitro exposure system, there is no known device which is capable of isolating the cell-matrices and reducing sources of contamination. Currently, the only available method to reduce cross contamination between wells on the VITROCELL is to increase the duration of purge air after an exposure event. This process not only slows the throughput of samples, but also introduces another, potentially, critical variable into the exposure conditions experienced by the cell matrices. Furthermore, clean air purges do not prevent methods of contamination that are the result of deposited debris or transfer from high concentration sources as a result of the cells being exposed to the ambient lab environment. Because the cover occludes each individual well and limits gas exchange between the wells, it has the potential to reduce the necessary purge time between sample sessions. Additionally, because of the quick deploy design, the well plate cover minimizes the time (e.g., under 30 seconds) that samples are left exposed to the ambient lab environment and other potential sources of contamination (dust, debris, contact transfer, etc.). The modular well plate cover also offers users a high degree of flexibility in regard to well plate prep and sample location. If a smaller sample size is required, the covers can be reduced in size to accommodate the new layout. If sample collection requires additional time or a non-standard layout is used, the cover caps can be deployed and removed individually, ensuring that cells stay isolated from one another for the full duration of sample collection. The well plate cover materials are designed to be both reusable and able to be decontaminated (autoclavable/chemical cleaning), ensuring that the covers are not themselves a source of contamination.

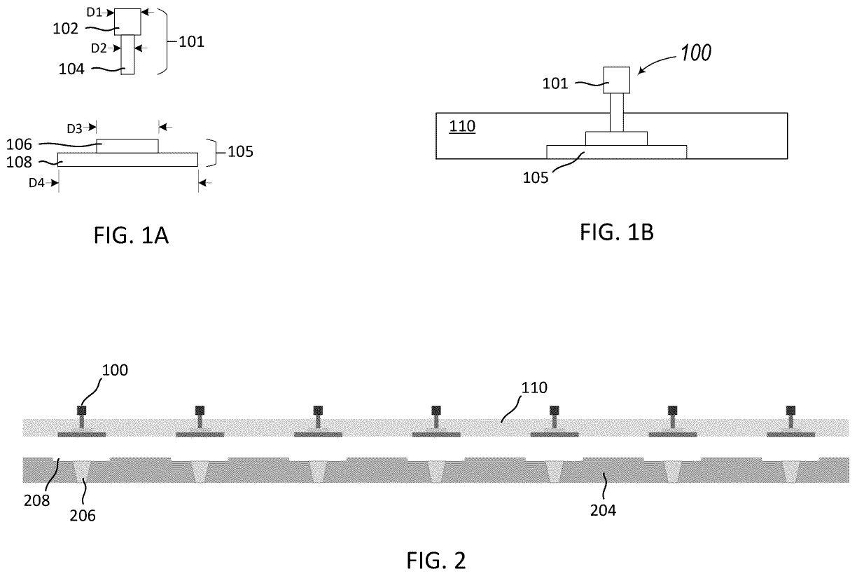

FIGS. 1A and 1B are cross sectional views illustrating a single well cover 100 consistent with the present disclosure. The well cover 100 includes a locking pin 101 coupled to a cover 105. The locking pin 101 may be constructed as a single piece, or as two pieces, a locking pin cap 102 and a magnetic locking pin 104. The cover 105 may be constructed as a single piece, or as two pieces, a cover magnet 106 and a cover cap 108.

In an embodiment, the single piece locking pin 101 may have a first pin section (equivalent to the locking pin cap 102) with a first pin diameter D1 and a second pin section (equivalent to the magnetic locking pin 104) with a second pin diameter D2. In an embodiment, the first pin section may have a height in the range of 0.20-0.30 inches, for example, 0.25 inches, and a diameter (or width, if a shape other than cylindrical) D1 in the range of 0.20-0.30 inches, for example, 0.25 inches, however other heights or diameters may be supported. In an embodiment, the second pin section may have a diameter (or width, if a shape other than cylindrical) D2 in the range of 0.120-0.130 inches, for example, 0.125 inches, and a height in the range of 0.70-0.80 inches, for example, 0.75 inches. In other embodiments, the magnetic locking pin 104 may have any other diameter (or width) and/or height as would be appropriate for the particular application.

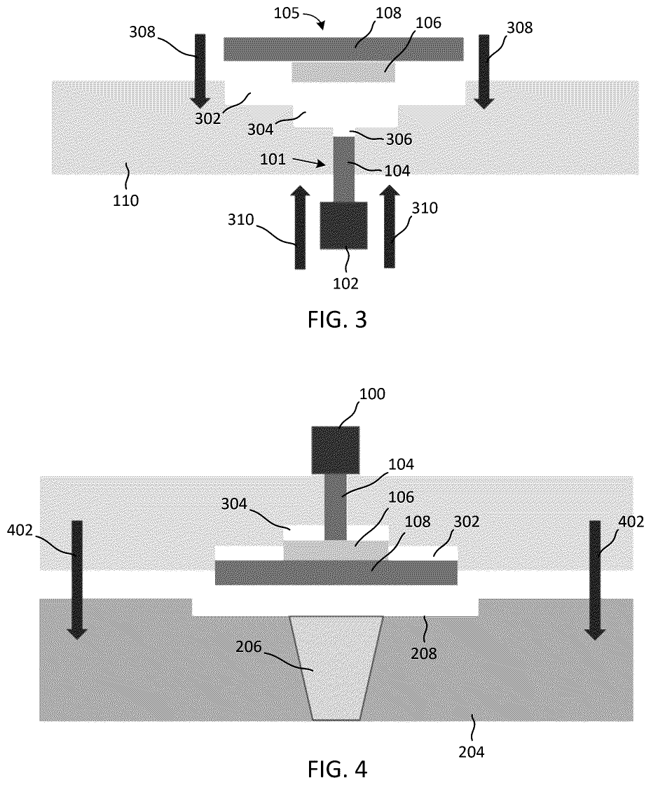

In embodiments where the locking pin 101 is comprised of two separate pieces, the locking pin cap 102 and the magnetic locking pin 104, the locking pin cap 102 may be cylindrical. In other embodiments, the locking pin cap 102 may be other shapes, e.g., a cube. In some embodiments, the outer surface of the locking pin cap 102 may contain a texture, e.g., a knurled finish, to make the locking pin cap 102 easier to grasp. In an embodiment, the locking pin cap 102 may be manufactured of steel, but any other appropriate material may be used as would be known to one skilled in the art. In an embodiment, the locking pin cap 102 may have a height in the range of 0.20-0.30 inches, for example, 0.25 inches, and a diameter (or width, if a shape other than cylindrical) D1 in the range of 0.20-0.30 inches, for example, 0.25 inches, however other heights or diameters may be supported. In an embodiment, the diameter of the locking pin cap 102 may be constrained by the diameter of the magnetic locking pin 104 such that the locking pin cap 102 may have a diameter greater than the magnetic locking pin 104 to prevent the locking pin cap 102 from passing through a hole 306 (FIG. 3) for the magnetic locking pin 104, thereby urging the combined locking pin assembly to remain attached to the modular well plate cover 110.

In an embodiment, the magnetic locking pin 104 may be cylindrical, although in other embodiments other shapes may be supported. The cylindrical shape, however, is optimal for ease of inserting the magnetic locking pin 104 into the modular well plate cover and to allow for enough freedom of movement for the cover cap 108 to lay flat when set down. The magnetic locking pin 104 has a first end coupled with the locking pin cap 102 and a second end distal from the first end and configured to magnetically couple with the cover 105. In an embodiment, the magnetic locking pin 104 may have a diameter (or width, if a shape other than cylindrical) D2 in the range of 0.120-0.130 inches, for example, 0.125 inches, and a height in the range of 0.70-0.80 inches, for example, 0.75 inches. In other embodiments, the magnetic locking pin 104 may have any other diameter (or width) and/or height as would be appropriate for the particular application. For example, in an embodiment for an instrument with larger diameter wells, the magnetic locking pin 104 may have a larger diameter (or width) and/or height to accommodate a larger well cover. In an embodiment, the magnetic locking pin 104 may be manufactured of steel, but any other appropriate material may be used as would be known to one skilled in the art. In embodiments where the magnetic locking pin 104 is not manufactured of steel or another magnetic material, the magnetic locking pin 104 may have a magnet disposed on or in the magnetic locking pin 104 at the end distal to the locking pin cap 102 to enable the magnetic locking pin 104 to magnetically couple with the cover magnet 106.

In an embodiment, the cover 105 may be a single part comprising a first cover section (equivalent to the cover magnet 106) with a first cover diameter D3 and a second cover section (equivalent to the cover cap 108) with a second cover diameter D4. The first cover diameter D3 of the first cover section may be less than or equal to the second cover diameter D4 of the second cover section.

In an embodiment, the second cover diameter D4 of the second cover section may be in the range of 1.30-1.70 inches, for example, 1.50 inches, and may have a height in the range of 0.20-0.30 inches, for example, 0.25 inches. In other embodiments, the second cover section may have any other diameter (or width) and/or height as would be appropriate for the particular application. For example, in an embodiment for an instrument with larger diameter wells, the second cover section may have a larger diameter (or width) and/or height to accommodate a larger well cover.

In an embodiment, the first cover section may have a height in the range of 0.062-0.125 inches. In other embodiments, the first cover section may have any other diameter (or width) and/or height as would be appropriate for the particular application. For example, in an embodiment for an instrument with larger diameter wells, the first cover section may have a larger diameter (or width) and/or height to accommodate a larger second cover section for a larger well in the target instrument. In an embodiment, the cover 105 may be manufactured of stainless steel, although in other embodiments other appropriate materials may be used as would be known to one skilled in the art.

In an embodiment, the one-piece cover 105 may be manufactured of a magnetic material or may have a separate magnet disposed in or on the surface of the one-piece cover cap/cover magnet that is configured to magnetically couple with the magnetic locking pin 104.

In an embodiment, the cover 105 may be comprised of two separate parts, the cover cap 108 and the cover magnet 106, coupled together. The cover magnet 106 may be cylindrical, although in other embodiments other shapes may be supported based, for example, on the shape of the opening in the well of the particular instrument for which the cover magnet 106 is intended. In an embodiment, the cover magnet 106 may have a first cover diameter (or width) D3 that is less than, or equal to, the diameter of the cover cap 108, and a height in the range of 0.062-0.125 inches. In other embodiments, the cover magnet 106 may have any other diameter (or width) and/or height as would be appropriate for the particular application. For example, in an embodiment for an instrument with larger diameter wells, the cover magnet 106 may have a larger diameter (or width) and/or height to accommodate a larger well cover. In an embodiment, the cover magnet 106 may be manufactured of stainless steel, although in other embodiments other appropriate materials may be used as would be known to one skilled in the art.

In some embodiments where the cover magnet 106 is not manufactured of steel or another magnetic material, the cover magnet 106 may have a magnet disposed in the surface of the cover magnet 106 distal from the surface of the cover magnet 106 that is coupled with the first surface of the cover cap 108 to enable the cover magnet 106 to magnetically couple with the magnetic locking pin 104.

In an embodiment, the cover 105 may be comprised of two separate parts, the cover cap 108 and the cover magnet 106, coupled together. The cover cap 108 may be cylindrical, although in other embodiments other shapes may be supported based, for example, on the shape of the opening in the well of the particular instrument for which the cover cap 108 is intended. In an embodiment, the cover cap 108 may have a second cover diameter (or width) D4 in the range of 1.30-1.70 inches, for example, 1.50 inches, and a height in the range of 0.20-0.30 inches, for example, 0.25 inches. In other embodiments, the cover cap 108 may have any other diameter (or width) and/or height as would be appropriate for the particular application. For example, in an embodiment for an instrument with larger diameter wells, the cover cap 108 may have a larger diameter (or width) and/or height to accommodate a larger well cover. In an embodiment, the cover cap 108 may be manufactured of stainless steel, although in other embodiments other appropriate materials may be used as would be known to one skilled in the art.

The cover cap 108 comprises a first surface configured to couple with the cover magnet 106, and a second essentially flat surface configured to cover the well of the instrument. In an embodiment, the cover cap 108 may be coupled to the cover magnet 106 using an appropriate adhesive. In other embodiments, the cover cap 108 may be coupled to the cover magnet 106 using any other appropriate method.

As with all the materials comprising the well cover 100, the material chosen for the locking pin 101, the locking pin cap 102, the magnetic locking pin 104, the cover 105, the cover magnet 106, and/or the cover cap 108 may be selected to be both reusable and able to be decontaminated (e.g., autoclavable, or chemical cleaning), ensuring that the covers are not themselves a source of contamination.

The well cover 100 may be assembled into the modular well plate cover 110. The construction and use of the modular well plate cover 110 is described in FIGS. 3-6.

FIG. 2 is a cross sectional view illustrating a plurality of well covers 100 loaded in the modular well plate cover 110, consistent with the present disclosure. Each of the plurality of well covers 100 are configured to align with a corresponding well 206 in the well plate 204 of the instrument. As is further shown in FIGS. 3-6, the well 106 further includes a well shoulder 208, which is the surface that the cover cap 108 of the well cover 100 is configured to couple with to seal the well 206.

FIGS. 3-6 are cross sectional views illustrating the assembly and placement of a plurality of well covers loaded in a modular well plate cover, consistent with the present disclosure.

In FIG. 3, the modular well plate cover 110 is inverted to allow for loading of the cover 105 into the first recess 302 and the second recess 304 in the modular well plate cover 110.

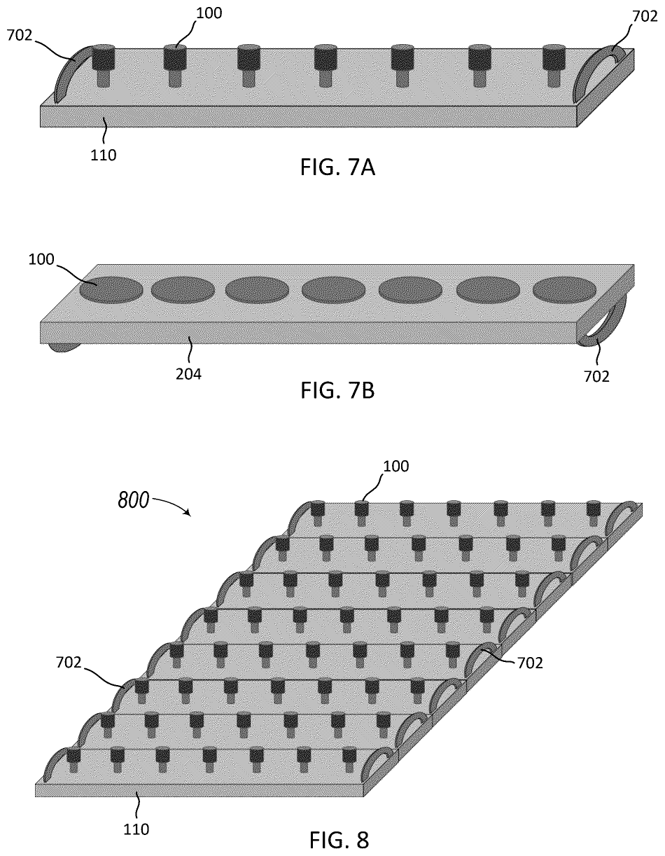

The modular well plate cover 110 consists of a flat bar, which has a plurality of recesses designed to house a series of well covers. In some embodiments, the modular well plate cover may include one or more handles 702 (FIGS. 7A and 7B) to provide for ease of handling the modular well plate cover. The first recess 302 is configured to accept the cover cap 108, and the diameter of the recess 302 is therefore greater than the diameter of the cover cap 108, but small enough to urge the cover cap 108 into a proper alignment with the well shoulder 208 of the well 206. The depth of the second recess 302 may be sufficient to prevent lateral movement of the cover cap 108 to maintain the proper alignment with the well 206.

The second recess 304 in the modular well plate cover 110 is configured to accept the cover magnet 106, and the diameter (or width) of the second recess 304 is therefore greater than the diameter of the cover magnet 106.

The modular well plate cover 110 also includes hole 306 to allow the magnetic locking pin 104 to pass through the modular well plate cover 110 to couple to the cover 105 when assembled to the modular well plate cover 110. In an embodiment, the hole 306 may have a diameter that is greater than or equal to the diameter D2 of the magnetic locking pin 104.

The cover 105 is loaded into the modular well plate cover 110 in the direction of arrows 308.

Either prior to loading the cover 105 into the modular well plate cover 110, or subsequent to loading the cover 105 into the modular well plate cover 110, the locking pin 101 is loaded into the modular well plate cover 110 from the opposite side of the modular well plate cover 110 from which the cover 105 is loaded into the modular well plate cover 110 by inserting the locking pin 101 into the hole 306 in the direction of arrows 310. Once both the locking pin 101 and the cover 105 have been inserted into the modular well plate cover 110, the locking pin 101 and the cover 105 may be magnetically coupled and therefore will remain loaded in the modular well plate cover 110 while the modular well plate cover 110 is further manipulated, i.e., to load additional well covers 100 or to install a complete modular well plate cover 110 into the instrument.

In FIG. 4 the loaded modular well plate cover 110 is lowered onto the well plate 204 on the instrument in the direction of arrows 402. The loaded modular well plate cover 110 is positioned such that the well covers 100 are aligned with the wells 206 and the well shoulders 208.

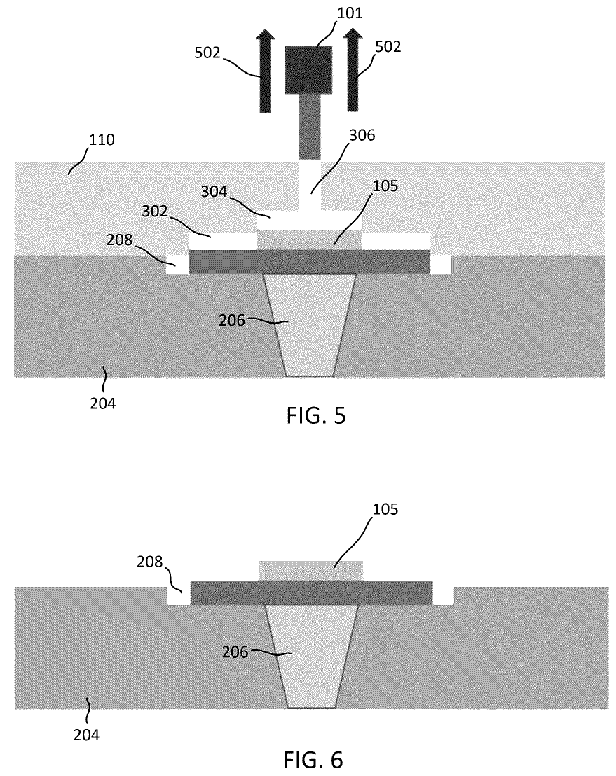

In FIG. 5, the modular well plate cover is positioned on the top surface of the well plate 204 with the well covers 100 properly aligned with the wells 206. In this operation, the locking pin 101 is removed from the well cover 100 by moving the locking pin 101 in the direction of arrows 502. As the locking pin 101 is extracted from the modular well plate cover 110, the cover 105 disengages from the locking pin 101 once the cover contacts the first recess 302 and/or the second recess 304 of the modular well plate cover 110, thereby decoupling the magnetic locking pin 104 from the cover magnet 106.

In FIG. 6, the modular well plate cover 110 has been removed from the well plate 204, leaving the cover 105 in place to seal the well 206.

FIGS. 7A and 7B are a top perspective view (FIG. 7A) and a bottom perspective view (FIG. 7B) of a plurality of well covers loaded in a modular well plate cover, consistent with the present disclosure. The modular well plate cover 110 in FIGS. 7A and 7B includes one or more optional handles 702 to assist in handling the modular well plate cover. In the example of FIGS. 7A and 7B, a plurality of well covers 100 are disposed in a modular well plate cover 110 ready to be installed in an instrument.

FIG. 8 is a perspective top view of a rack 800 of modular well plate covers 110 loaded with a plurality of well covers 100, consistent with the present disclosure. In the example of FIG. 8, a plurality of modular well plate covers 110 are arranged in a rack configuration to allow for full wet plate coverage in the instrument in a single operation.

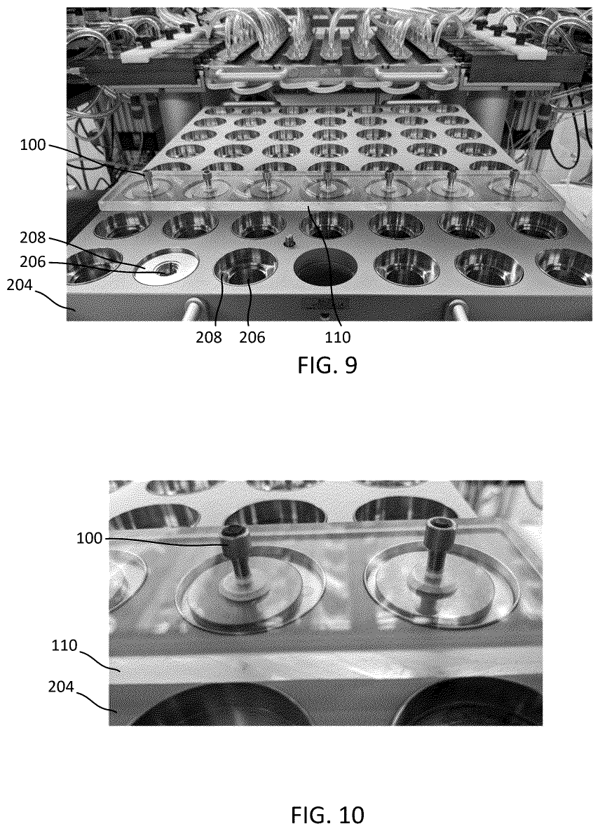

FIG. 9 is an illustration of a modular well plate cover 110 of well covers 100 in position for assembly to a well plate 204.

FIG. 10 is a close-up illustration of two well covers 100 in a modular well plate cover 110 in position for assembly to a well plate 204.

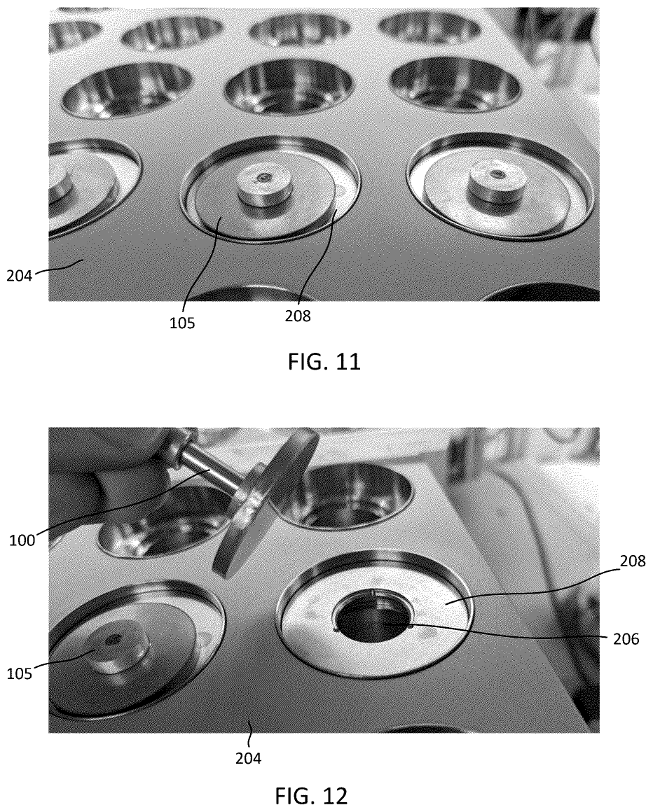

FIG. 11 is a close-up illustration of two covers 105 inserted into a well plate 204.

FIG. 12 is an illustration of a well cover 100 being removed from a well plate 204.

According to one aspect of the disclosure there is thus provided a contamination guard for an in vitro exposure system. The contamination guard includes a plurality of well covers. Each of the plurality of well covers further includes: a locking pin; and a cover, the cover configured to cover a well in the in vitro exposure system to isolate the well from contamination; and a modular well plate cover, the modular well plate cover configured to align the plurality of well covers over a well plate of the in vitro exposure system.

According to another aspect of the disclosure, there is thus provided a modular cross contamination guard system. The system includes: a plurality of well covers; and a modular well plate cover, the modular well plate cover configured to removably couple to the plurality of well covers and to align each of the plurality of well covers with a corresponding well in a well plate of an instrument.

The present disclosure is not limited in its application to the details of construction and the arrangement of components set forth in the following description or illustrated in the drawings. The examples described herein may be capable of other embodiments and of being practiced or being carried out in various ways. Also, it may be appreciated that the phraseology and terminology used herein is for the purpose of description and should not be regarded as limiting as such may be understood by one of skill in the art. Throughout the present disclosure, like reference characters may indicate like structure throughout the several views, and such structure need not be separately discussed. Furthermore, any particular feature(s) of a particular exemplary embodiment may be equally applied to any other exemplary embodiment(s) of this disclosure as suitable. In other words, features between the various exemplary embodiments described herein are interchangeable, and not exclusive.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and C; B and C; or A, B and C.

The term “coupled” as used herein refers to any connection, coupling, link, or the like by which signals carried by one system element are imparted to the “coupled” element. Such “coupled” devices, or signals and devices, are not necessarily directly connected to one another and may be separated by intermediate components or devices that may manipulate or modify such signals.

Unless otherwise stated, use of the word “substantially” may be construed to include a precise relationship, condition, arrangement, orientation, and/or other characteristic, and deviations thereof as understood by one of ordinary skill in the art, to the extent that such deviations do not materially affect the disclosed methods and systems. Throughout the entirety of the present disclosure, use of the articles “a” and/or “an” and/or “the” to modify a noun may be understood to be used for convenience and to include one, or more than one, of the modified noun, unless otherwise specifically stated. The terms “comprising”, “including” and “having” are intended to be inclusive and mean that there may be additional elements other than the listed elements.

Although the methods and systems have been described relative to a specific embodiment thereof, they are not so limited. Obviously, many modifications and variations may become apparent in light of the above teachings. Many additional changes in the details, materials, and arrangement of parts, herein described and illustrated, may be made by those skilled in the art.

What is claimed is:

1. A contamination guard for an in vitro exposure system, the contamination guard comprising:
a plurality of well covers, each of the plurality of well covers further comprising:
a locking pin; and
a cover, the cover configured to cover a well in the in vitro exposure system to isolate the well from contamination; and
a modular well plate cover, the modular well plate cover configured to align the plurality of well covers over a well plate of the in vitro exposure system.

2. The contamination guard of claim 1, wherein the locking pin is a cylinder, and has a first pin section that has a first pin diameter and a second pin section that has a second pin diameter, and wherein the first pin diameter is larger than the second pin diameter.

3. The contamination guard of claim 2, wherein the locking pin further comprises:
a magnet disposed in an end of the second pin section of the locking pin that is distal from the first pin section of the locking pin.

4. The contamination guard of claim 1, wherein the locking pin further comprises:
a locking pin cap; and
a magnetic locking pin.

5. The contamination guard of claim 4, wherein the locking pin cap is a cylinder that has a first pin diameter and the magnetic locking pin has a second pin diameter, and wherein the first pin diameter is larger than the second pin diameter.

6. The contamination guard of claim 4, wherein the magnetic locking pin is constructed of a magnetic material.

7. The contamination guard of claim 4, wherein the magnetic locking pin further comprises:
a magnet disposed in an end of the magnetic locking pin distal from the locking pin cap.

8. The contamination guard of claim 1, wherein the cover is a cylinder, and has a first cover section that has a first cover diameter and a second cover section that has a second cover diameter, and wherein the first cover diameter is larger than the second cover diameter.

9. The contamination guard of claim 8, wherein the cover further comprises:
a magnet disposed in a surface of the first cover section that is distal from the second cover section.

10. The contamination guard of claim 1, wherein the cover further comprises:
a cover magnet; and
a cover cap.

11. The contamination guard of claim 10, wherein the cover magnet is constructed of a magnetic material.

12. The contamination guard of claim 10, wherein the cover magnet further comprises:
a magnet disposed in a surface of the cover magnet that is distal from the cover cap.

13. The contamination guard of claim 1, wherein the modular well plate cover further comprises:
a flat bar; and
a plurality of recesses configured to align each of the well covers with a plurality of wells in the well plate of the in vitro exposure system.

14. The contamination guard of claim 13, wherein each of the plurality of wells includes:
a first recess configured to hold a first cover section of the cover; and
a second recess configured to hold a second cover section of the cover, wherein the second recess further configured to align each of the plurality of well covers with a well in the well plate of the in vitro exposure system.

15. A modular cross contamination guard system, the system comprising:
a plurality of well covers; and
a modular well plate cover, the modular well plate cover configured to removably couple to the plurality of well covers and to align each of the plurality of well covers with a corresponding well in a well plate of an instrument.

16. The system of claim 15, wherein each of the plurality of well covers further comprises:
a locking pin; and
a cover, the cover configured to cover a well in the instrument.

17. The system of claim 16, wherein the locking pin is magnetically coupled to the cover.

18. The system of claim 16, wherein the locking pin is a cylinder, and has a first pin section that has a first pin diameter and a second pin section that has a second pin diameter, and wherein the first pin diameter is larger than the second pin diameter.

19. The system of claim 16, wherein the cover is a cylinder, has a first cover section that has a first cover diameter and a second cover section that has a second cover diameter, and wherein the first cover diameter is larger than the second cover diameter.

20. The system of claim 15, wherein the modular well plate cover further comprises:
one or more handles, the one or more handles configured to provide for ease of handling the modular well plate cover.
